(19) United States
(12) Patent Application Publication
(10) Pub. No.: US 2013/0072959 A1
(43) Pub. Date: Mar. 21, 2013

(54) Non-Fragmenting Low Friction Bioactive Absorbable Coils for Brain Aneurysm Therapy

(71) Applicants: Benjamin M. Wu, San Marino, CA (US); Fernando Vinuela, Los Angeles, CA (US); Yuichi Marayama, Los Angeles, CA (US); Yuhuan Xu, Santa Monica, CA (US)

(72) Inventors: Benjamin M. Wu, San Marino, CA (US); Fernando Vinuela, Los Angeles, CA (US); Yuichi Marayama, Los Angeles, CA (US); Yuhuan Xu, Santa Monica, CA (US)

(73) Assignee: THE REGENTS OF THE UNIVERSITY OF CALIFORNIA, Oakland, CA (US)

(21) Appl. No.: 13/552,616

(22) Filed: Jul. 18, 2012

Related U.S. Application Data
(63) Continuation of application No. 11/467,847, filed on Aug. 28, 2006, now abandoned.
Application No. 11/467,847 is a continuation-in-part of application No. 11/198,587, filed on Aug. 5, 2005.
Application No. 11/198,587 is a continuation-in-part of application No. 09/785,743, filed on Feb. 16, 2001, now Pat. No. 7,070,607.
Application No. 09/785,743 is a continuation-in-part of application No. 09/406,306, filed on Sep. 27, 1999, now Pat. No. 6,423,085.
Application No. 09/406,306 is a continuation of application No. PCT/US1999/001790, filed on Jan. 27, 1999.
(60) Provisional application No. 60/072,653, filed on Jan. 27, 1998.

(51) Int. Cl. A61M 29/00

(52) U.S. Cl. CPC A61M 29/00
USPC 606/200

(57) ABSTRACT

Non-fragmenting low friction bioactive absorbable coils are disclosed that improve long-term anatomic results in the endovascular treatment of intracranial aneurysms. The coils are composed of at least one biocompatible and bioabsorbable polymer. The coils are then coated with a polymer to reduce the friction. The coating can contain drugs, such as growth factors, and can be used to accelerate histopathologic transformation in aneurysms. The coil can be a polymer such as polyglycolic acid (PGA), poly-L-lactic acid (PLLA), polycaprolactive, poly-L-lactide, polydioxanone, polycarbonates, polyanhydrides, polyglycolic acid/poly-L-lactic acid copolymers, polyhydroxybutyrate/hydroxyvalerate copolymers, or combinations thereof.

### RELATED APPLICATION

This application is a continuation application of U.S. patent application Ser. No. 11/467,847, filed Aug. 28, 2006, which is a continuation-in-part application of U.S. patent application Ser. No. 11/198,587, filed Aug. 5, 2005, which is a continuation-in-part application of U.S. patent application Ser. No. 09/785,743, filed Feb. 16, 2001, now U.S. Pat. No. 7,070,607, which is a continuation-in-part application of U.S. patent application Ser. No. 09/406,306 filed Sep. 27, 1999, now U.S. Pat. No. 6,423,085, which is a continuation of PCT International Application No. PCT/US99/01790, filed Jan. 27, 1999, which claims the benefit of which is related to U.S. Provisional Patent Application Ser. No. 60/072,653, filed Jan. 27, 1998, and all of which are incorporated herein by reference in their entirety.

### GOVERNMENT INTEREST

This invention was made with government support under Government Grant No. NS42316 awarded by the National Institute of Health. The government has certain rights in the invention.

### FIELD OF INVENTION

The present invention relates generally to the field of surgical and endovascular interventional apparatus and in particular to drug-eluding implants for occlusion of vessels or aneurysms.

### BACKGROUND

Subarachnoid hemorrhage from intracranial aneurysm rupture remains a devastating disease. Endovascular occlusion of ruptured and unruptured intracranial aneurysms using Guglielmi detachable coil (GDC) technology has recently gained worldwide acceptance as a less-invasive treatment alternative to standard microsurgical clipping. However, critical evaluation of the long-term anatomical results of aneurysms treated with metal coils shows three limitations. First, compaction and aneurysm recanalization can occur. This technical limitation is more often seen in small aneurysms with wide necks and in large or giant aneurysms. Second, tight packing of metal coils in large or giant aneurysms may cause increased mass effect on adjacent brain parenchyma and cranial nerves. Third, the standard platinum metal coil is relative biological inert. Recent reports of methods to favorably enhance the biological activity of metal coils highlight the increased interest in finding innovative solutions to overcome these present biological limitations of the conventional metal coil system.

Recent animal investigations and post-mortem human histopathologic studies have provided valuable information on the histopathological changes occurring in intracranial aneurysms in patients treated with metal coils. Both animal and human studies support the hypothesis that a sequential bio-cellular process occurs in the aneurysm leading to the development of organized connective tissue after metal coil placement and altered hemodynamics. It has been postulated that the histological changes observed in an aneurysm after metal coil occlusion follow the general pattern of wound healing in a vessel wall. In support of metal coil-induced favorable histopathological transformation, in the largest post-mortem study reported, some aneurysms packed with metal coils demonstrated reactive fibrosis in the body of the aneurysm and anatomic exclusion of the orifice within six weeks after treatment. Moreover, the use of polymer coated coils instead of metal coils results in granulation tissue formation around the coils. Thus, all the current coils lack robust biological response. Therefore, a need exists for coils and methods for brain aneurysm therapy that promote an inflammatory response and healing of the aneurysm with reduction of its mass effect.

### SUMMARY

The present invention provides methods, compounds, and compositions for the treatment of a brain aneurysm. The compositions comprise an absorbable coil that is non-fragmenting and has low friction. The compositions can further comprise a drug, such as a modulator of vascular permeability, for the treatment or prevention of diseases in a subject in need thereof.

In one aspect of the invention, endovascular apparatus comprising a biocompatible and bioabsorbable polymer, and a coating on the polymer coils wherein the coating reduces friction is provided. The biocompatible and bioabsorbable polymer can be polyglycolic acid (PGA), poly-L-lactic acid (PLLA), polycaprolactive, poly-L-lactide, polydioxanone, polycarbonates, polyanhydrides, polyglycolic acid/poly-L-lactic acid copolymers, polyhydroxybutyrate/hydroxyvalerate copolymers, or combinations thereof, and the coating can be polylactide/polyglycolide copolymer (PLGs), caprolactone, calcium stearoyl lactylate, caprolactone/glycolide copolymer, or combinations thereof. In addition, the coating can include drugs, such as growth factor vascular endothelial growth factor (VEGF), basic fibroblast growth factor (b-FGF), transforming growth factors (TGF), platelet-derived growth factors (PDGF), or mixtures thereof.

In another aspect, the invention provides polymer coils comprising a biocompatible and bioabsorbable polymer, and a sandwich coating on the polymer coils wherein the sandwich coating comprises at least a first coat and a second coat and wherein the sandwich coating reduces friction. The biocompatible and bioabsorbable polymer can be polyglycolic acid (PGA), poly-L-lactic acid (PLLA), polycaprolactive, poly-L-lactide, polydioxanone, polycarbonates, polyanhydrides, polyglycolic acid/poly-L-lactic acid copolymers, polyhydroxybutyrate/hydroxyvalerate copolymers, or combinations thereof. The first coat and the second coat can be polylactide/polyglycolide copolymer (PLGs), caprolactone, calcium stearoyl lactylate, caprolactone/glycolide copolymer, or combinations thereof. In addition, the first coat can include drugs, such as growth factor vascular endothelial growth factor (VEGF), basic fibroblast growth factor (b-FGF), transforming growth factors (TGF), platelet-derived growth factors (PDGF), or mixtures thereof.

These and other aspects of the present invention will become evident upon reference to the following detailed description. In addition, various references are set forth herein which describe in more detail certain procedures or compositions, and are therefore incorporated by reference in their entirety.

### BRIEF DESCRIPTION OF DRAWINGS

FIG. 1 illustrates the granulation of tissue formation around the polymer coils.

FIG. 2 illustrates the hypothesis of how granulation of tissue formation occurs around polymer coils.

FIG. 3 illustrates the effect of coating the polymer coils on the immune response.

FIG. 4 illustrates one method of coating the coils.

FIG. 5 shows the TEM figures of uncoated polymer coils and coated polymer coils.

FIG. 6 illustrates a polysorb polymer fiber, a polysorb polymer fiber with a single coating, and a polysorb polymer fiber with a sandwich coating.

### DETAILED DESCRIPTION

### I. Definitions

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. The practice of the present invention will employ, unless otherwise indicated, conventional methods of protein chemistry, biochemistry, and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Remington's Pharmaceutical Sciences, 18th Edition (Easton, Pa.: Mack Publishing Company, 1990).

All publications, patents and patent applications cited herein, whether supra or infra, are hereby incorporated by reference in their entirety.

The terms “effective amount” or “pharmaceutically effective amount” refer to a nontoxic but sufficient amount of the agent to provide the desired biological result. That result can be reduction and/or alleviation of the signs, symptoms, or causes of a disease, or any other desired alteration of a biological system. For example, an “effective amount” for therapeutic uses is the amount of the composition comprising a drug disclosed herein required to provide a clinically significant modulation in the symptoms associated with vascular permeability. An appropriate “effective amount” in any individual case may be determined by one of ordinary skill in the art using routine experimentation.

As used herein, the terms “treat” or “treatment” are used interchangeably and are meant to indicate a postponement of development of a disease associated with vascular permeability and/or a reduction in the severity of such symptoms that will or are expected to develop. The terms further include ameliorating existing symptoms, preventing additional symptoms, and ameliorating or preventing the underlying metabolic causes of symptoms.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

By “physiological pH” or a “pH in the physiological range” is meant a pH in the range of approximately 7.2 to 8.0 inclusive, more typically in the range of approximately 7.2 to 7.6 inclusive.

The term “polymer” is defined as being inclusive of homopolymers, copolymers, and oligomers. The term “homopolymer” refers to a polymer derived from a single species of monomer. The term “copolymer” refers to a polymer derived from more than one species of monomer, including copolymers that may be obtained by copolymerization of two monomer species, those that may be obtained from three monomers species (“terpolymers”), those that may be obtained from four monomers species (“quaterpolymers”), etc.

The term “poly(lactic acid-co-glycolic acid)” or “PLGA” refers to a copolymer formed by co-polycondensation of lactic acid, HO—CH(CH₃)—COOH, and glycolic acid, HO—CH₂—COOH.

The term “low friction” refers to the minimization of frictional forces between neighboring coils; and between coil and catheter; as the coil is either advanced (pushed) or retracted (pulled) during treatment.

As used herein, the term “subject” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or gender.

### II. Modes of Carrying Out the Invention

The invention provides compositions and methods for the treatment of brain aneurysms. The compositions comprise an absorbable coil that is non-fragmenting and has low friction, and can further comprise a drug. The compositions are used in methods for the treatment or prevention of brain aneurysms in a subject in need thereof.

The use of absorbable polymeric materials in biomedical engineering has dramatically increased during the past decade because of their interesting and well-studied properties. Bioabsorbable polymeric materials do not elicit intense chronic foreign body reaction because they are gradually absorbed and do not leave residua at the implantation site. In general, a faster degrading bioabsorbable polymeric material will result in a stronger inflammatory reaction. By altering polymer composition and therefore degradation times, intravascular inflammatory reactions can be controlled. Some bioabsorbable polymeric material is capable of regenerating tissue through the interaction of immunologic cells such as macrophages. Bioabsorbable polymeric material as an embolic material for the treatment of the intracranial aneurysms offers three main advantages that are capable of overcoming the current anatomical limitations of the metal coil system. First, bioabsorbable polymeric material stimulates mild to strong cellular infiltration and proliferation in the process of degradation that can accelerate fibrosis within aneurysms. Accelerated fibrosis within the aneurysm leads to stronger anchoring of coils. Second, organized connective tissue filling an aneurysm tends to retract over time due to maturation of collagen fibers (scar tissue). This connective tissue retraction can reduce aneurysm size and can decrease aneurysm compression on brain parenchyma or cranial nerves. Third, coil thrombogenicity is an important property of an embolic device. Bioabsorbable polymeric material can accelerate aneurysm healing with less thrombogenicity. Other advantages of bioabsorbable polymeric material include their shape versatility, cheaper cost of manufacture, and optional use as a drug delivery vehicle. Various proteins, cytokines, and growth factors can be implanted in bioabsorbable polymeric material and slowly delivered during bio-absorption. A drug delivery system using bioabsorbable polymeric material provides great potential for controlled healing of aneurysms.

The coil can be any type of coil known in the art, such as, for example, a Guglielmi detachable coil (GDC). The coil can be coated with an absorbable polymeric material to improve long-term anatomic results in the endovascular treatment of intracranial aneurysms. The coil can further be coated to decrease friction to decrease the granulation tissue formation around the coils. In one aspect of the invention, the coat comprises at least one biocompatible and bioabsorbable polymer and growth factors, and is used to accelerate histopathologic transformation of unorganized clot into fibrous connective tissue in aneurysms.

An endovascular cellular manipulation and inflammatory response can be elicited from implantation of the disclosed non-fragmenting, low-friction bioactive absorbable coils in a vascular compartment or any intraluminal location. Thrombogenicity of the biocompatible and bioabsorbable polymer can be controlled by the composition of the polymer, namely proportioning the amount polymer and copolymer in the coil or implant. The coil can further comprise a growth factor or more particularly a vascular endothelial growth factor, a basic fibroblast growth factor or other growth factors. The biocompatible and bioabsorbable polymer can be at least one polymer selected from the group consisting of polyglycolic acid (PGA), poly-L-lactic acid (PLLA), polycaprolactive, poly-L-lactide, polydioxanone, polycarbonates, polyanhydrides, polyglycolic acid/poly-L-lactic acid copolymers, and polyhydroxybutyrate/hydroxyvalerate copolymers, or combinations thereof.

Accelerating and modulating the aneurysm scarring process with bioactive materials overcomes the present long-term anatomic limitations of the metal coil systems, and the polymer coated coil systems. Bioabsorbable polymers or proteins can be manufactured to have mechanical properties favorable for endovascular placement. Certain polymers and proteins can be constructed and altered to regulate adjacent tissue and cellular reaction. Moreover, selected polymers or proteins can also be used as delivery vehicles (e.g., continuous local delivery of growth factors). Bioabsorbable polymeric materials, such as PGA, PLLA, and polyglycolic/poly-L-lactic acid copolymers, are well-studied biocompatible substances that have been used in tissue engineering applications. Bioabsorbable polymeric materials promote cellular reactions during their biological degradation. The degree of tissue reaction induced by bioabsorbable polymeric materials can be controlled by selecting polymer composition. Bioabsorbable polymeric materials can be utilized as a new bioabsorbable embolic material for the endovascular treatment of intracranial aneurysms. Compared to metal coils, bioabsorbable polymeric materials offer the advantages of accelerated aneurysm scarring and negative mass effect.

The coils can be metallic or nonmetallic coils, or can be any biocompatible material. Thus, the coils can be platinum, biocompatible plastics, or any bioabsorbable material. In one aspect, the coils can be composed of an inner core of platinum wire and an outer braid of bioabsorbable polymeric materials. In general threads of bioabsorbable polymeric materials in any form can be attached in any manner to the platinum wire or coil.

In one aspect of the invention, non-fragmenting, low-friction, bioactive absorbable polymer coils are used to control thrombosis or accelerate wound healing of the brain aneurysms for which platinum coils sometimes have often proven unsatisfactory. The bioactive absorbable polymer coils of the invention are non-fragmenting and low friction coils. Typically, successful coil deployment involves the opposing requirements of a strong junction that can quickly detach on demand. Besides limiting both the final coil density and the surgical approach, excessive friction would also increase the risk of coil deformation, failure, or malfunction during pushing and pulling. The typical pushing and pulling forces required to advance and retract a coil, respectively, into the aneurysm generally increases with increasing number of coils in the aneurysm, and with increasing tortuosity of the vascular system (due to intracatheter friction). If the coil-catheter friction is high, the latter friction is amplified and the resultant push force may cause the weakest link of the coil device to deform or even fail (typically the detachment zone). Excessive pulling forces can also induce unraveling or fracture of the previously placed coils. Therefore it is highly desirable to minimize the friction of aneurismal coils.

In another aspect of the invention, methods are provided for drug delivery using non-fragmenting, low-friction, bioactive absorbable polymer coils in combination with growth factors such as vascular endothelial growth factor (VEGF), basic fibroblast growth factor (bFGF) or other growth factors which promote long lasting effect of the wound healing.

The non-fragmenting, low-friction, bioactive absorbable polymer coils of the invention are useful for treating giant brain aneurysms to prevent the mass effect on the brain parenchyma or cranial nerves by shrinkage of scaring aneurysm.

In one aspect of the invention, the coil is a braided suture coated with a polymer to provide the non-fragmenting, low-friction, bioactive absorbable polymer coils of the invention. The braided suture can be fabricated using the methods and apparatus disclosed in the co-pending, co-owned PCT application titled “Oriented Polymer Fibers and Methods for Fabricating Thereof,” filed on Mar. 31, 2005, and published as WO 05/096744. The apparatus disclosed in WO 05/096744 can be used to make the polymer coils of the invention. The apparatus uses polymer dispersion where a solid polymer can be dispersed in the liquid dispersal phase using any standard dispersing method.

The disperse polymer phase can include a polymer or a polymer blend comprising a plurality of polymers. Any polymer capable of forming fibers can be used, particularly polar polymers capable of providing fibers with piezoelectricity, pyroelectricity, and ferroelectricity. Examples of such polymers that can be used include of polyglycolic acid (PGA), poly-L-lactic acid (PLLA), polycaprolactive, poly-L-lactide, polydioxanone, polycarbonates, polyanhydrides, polyglycolic acid/poly-L-lactic acid copolymers, polyhydroxybutyrate/hydroxyvalerate copolymers, or combinations thereof. Those having ordinary skill in the art may select other fiber-forming polymers.

Instead of using a solid polymer, if desired, a polymer solution can be used for dispersal in the liquid dispersal phase. To prepare the polymer solution, the polymer can be dissolved in a solvent. Any suitable solvent can be selected provided the selected solvent is immiscible with the liquid dispersal phase. A blend comprising a plurality of individual polymers can be used for making the polymer solution, so long as each individual polymer in the blend is soluble in the selected solvent, or when each individual polymer in the blend is pre-dissolved in a selected solvent, that the mixture of selected solvents form a solution.

The liquid phase dispersal phase comprises one or a plurality of liquids. Any suitable liquid(s) can be used for making the liquid dispersal phase as known to those having ordinary skill in the art, so long as the liquid(s) used for making the liquid dispersal phase cannot be true solvent(s) for any polymer that is present in the disperse phase.

The liquid dispersal phase can optionally contain various additives, for example, the additives capable of providing better control of solubility, charge, viscosity, surface tension, evaporation, boiling point, refractive index, to influence the final chemical, physical, and biological properties of the resultant fibers. One kind of additives that can be used includes a surfactant, the use of which is intended to facilitate the making of the dispersion. Any commonly used surfactant(s) can be utilized. Standard ratios between the quantities of the liquid dispersal phase and the surfactant can be used.

Another kind of additive that can be used in the liquid dispersal phase includes compounds designed to decrease the stability of the metastable dispersion. For example, a sodium chloride solution can be used for this purpose. It may be also desirable to be able to increase charge density on the surface of polymeric fibers to produce 3-dimension oriented fiber mats using polymers with little or no polarity. To that end, multi-valent cations or anions can be added to the polymeric dispersion.

In some embodiments it may be desirable to make the final polymer fiber biologically active. To that end, biologically active molecules can be added to the liquid dispersal phase. When the process of fabricating the polymer fibers is complete, the biologically active molecules are expected to be present in the final polymer fiber. Any biologically active substance can be used as the source of biologically active molecules. Representative examples include laminin and growth factors such as IGF (insulin-like growth factors), TGF (transforming growth factors), FGB (fibroblast growth factors), including b-FGF (basic fibroblast growth factors), EGF (epidermal growth factors), VEGF (vascular endothelial growth factors), BMP (bone morphogenic proteins), PDGF (platelet-derived growth factors), or combinations thereof. These growth factors are well known and are commercially available.

If it is desirable to incorporate the biologically active molecules within the bulk of the fiber, surfactants can help increase the solubility of the biologically active molecules within the polymer liquid phase, particularly when biologically active molecules that are being incorporated into the fiber have low water solubility, such as hydrophobic drugs or steroids, etc.

The metastable polymer dispersion is made and placed into the dispenser described in WO 05/096744, and the metastable polymer dispersion can be electrically pulled through the orifice to form polymer fiber that can be collected on the collector. The polymer fiber that can be collected can be a 3-dimensional oriented fiber. Thus, for example, the fiber can be a co-polymer of PGA (93%) and PLLA (7%).

The fiber thus obtained can be coated to provide the low friction coils. The coating can be up to 100 μm thick. Thus, the average thickness of the coating is preferably 100 μm or less, although spots with a thickness of more than 100 μm, occasioned by fluctuations in the coating process, are contemplated to be within the scope of the present invention. Thus, the coating can be about 0.01 μm to about 100 μm thick, preferably about 1 μm to about 95 μm, or more preferably about 10 μm to about 90 μm thick, or any thickness in between.

The coating can be a polymer preferably selected from the group comprising lactones, poly-α-hydroxy acids, polyglycols, polytyrosine carbonates, starch, gelatins, cellulose as well as blends and interpolymers containing these components. Particularly preferred among the poly-α-hydroxy acids are the polylactides, polyglycol acids, and their interpolymers. Thus, the coat can be caprolactone/glycolide copolymer or calcium stearoyl lactylate. Calcium stearoyl lactylate degrades into stearic and lactic acids. The coat can also be acidic polyesters, such as a mixture of PLGA and hydroxyacetic acid (about equivalent molar ratios), or polyester anhydrides such as glycolic acid, lactic acid, or sebacic acid polymers.

The coating may contain additional pharmaceutically active agents, such as osteoinductive or biocidal or anti-infection substances. Suitable osteoinductive substances include, for example, growth factors whose proportion of the total weight of the coating is preferably 0.1 to 10% by weight or, more preferably, 0.5 to 8% by weight and, most desirably, 1 to 5% by weight. This weight percentage relates to the net amount of the active agent, without counting any pharmaceutical carrier substances.

In one aspect of the invention, the polymer fiber can be coated with a single surface coating where the surface coating contains the drug. In another aspect of the invention, the polymer fiber can be sandwich coated, where the suture is coated with two surface coats where only one of the coats contains the drugs. Preferably, the polymer fiber is sandwich coated where the first coat contains the drug, and the first coat is coated again with PLGS.

### Modes for Carrying out the Invention

The implants of the invention may be placed within body lumens, e.g., blood vessels, Fallopian tubes, etc., of any mammalian species, including humans. The implant coils are made of biocompatible and bioabsorbable polymers or proteins.

To achieve radioopacity, the bioabsorbable polymer coils may be coated or mixed with radioopaque materials such as tantalum or platinum. The bioabsorbable polymer or protein itself may be mounted or coated onto coils or wires of metals such as platinum or nitinol.

Preferred growth factors for use in the invention are the naturally occurring mammalian angiogenic growth such as VEGF, or b-FGF. Mixtures of such growth factors may also be used if desired.

The non-fragmenting, low-friction, bioactive absorbable polymer coils of the invention can be placed within the body lumen, vascular system or vessels using procedures well known in the art. Generally, the desired site within the vessel is accessed with a catheter. For small diameter torturous vessels the catheter may be guided to the site by the use of guide wires. Once the site has been reached, the catheter lumen can be cleared by removing guide wire. In the case of polymer occlusion coils, the coils are loaded by means of a pusher wire. The coils can be attached to the distal end of the pusher via a cleavable joint (e.g., a joint that is severable by heat, electrolysis, electrodynamic activation or other means) or a mechanical joint that permits the implant to be detached from the distal end of the pusher wire by mechanical manipulation. Alternatively, the coils can be freed and detached from the pusher wire, simply pushed through the catheter and expelled from the distal end of the catheter.

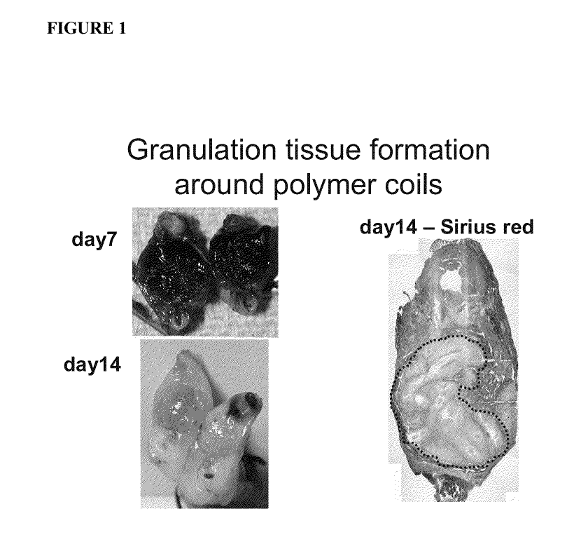

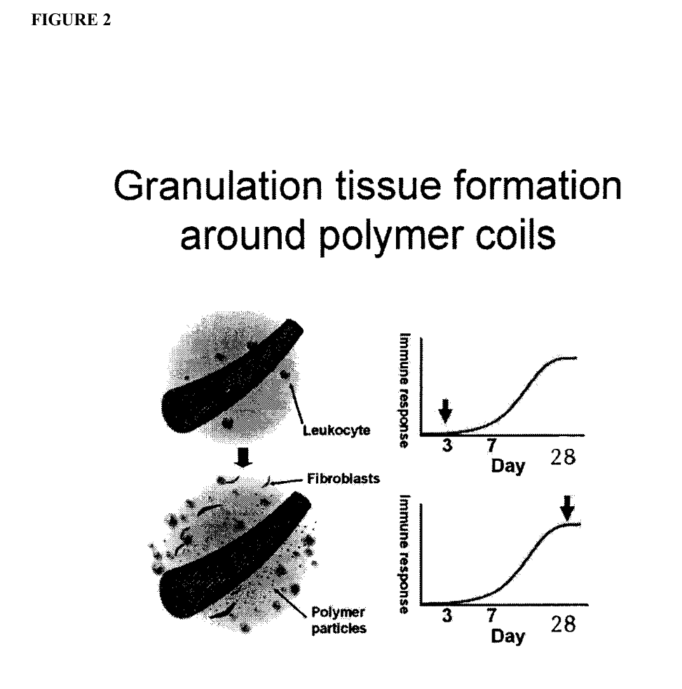

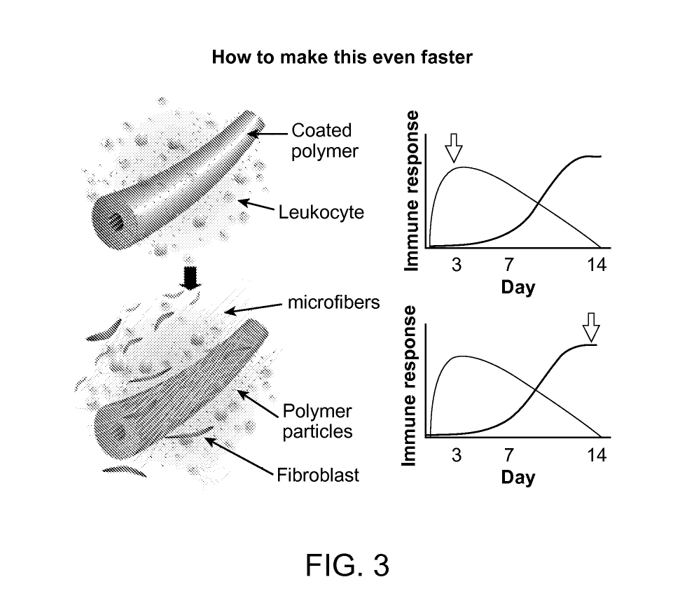

The implantation of polymer coils results in the formulation of granulation tissue around the coils as shown in FIG. 1. Without being bound to theory, it is hypothesised that the granulation of tissue formation around polymer coils occurs due to the polymer degradation products surrounding the coils that attract inflammatory and repair cells (FIG. 2). The upper left image in FIG. 2 illustrates initial recruitment of inflammatory cells to the coil at day 3, while the corresponding graph in the upper right image in FIG. 2 shows that minimal granulation tissues are deposited at day 3, as the aneurysm is filled with clot, cellular infiltrates, and coils. By day 28, the polymer degradation products are released from the coils and by this time repair cells such as circulating stem cells and fibroblasts have infiltrated the aneurysm (lower left of FIG. 2) and synthesized ample granulation tissues (lower right of FIG. 2) The effect of coating the polymer coils is illustrated in FIG. 3, where the non-fragmenting low friction bioactive absorbable coils of the invention elicit a more rapid inflammatory response, leading to and more robust deposition of granulation tissues, and ultimately faster recovery. This is accomplished by the release of pro-inflammatory biochemicals from the coating material. Since the thickness of this pro-inflammatory material is thin, and is limited only to the outermost surface of the coil, the stimulation is limited to the early, initial stages of wound healing, and will not continue to elicit prolonged inflammation, as illustrated by the single-burst curve (arrow) in the upper right graph in FIG. 3. The initial stimulation is enough to accelerate the granulation tissue response by day 14 (arrow; lower right graph in FIG. 3).

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1

### Fabrication of Fibers Incorporating Biologically Active Molecules

A solution of PLGA in chloroform was mixed with NaCl water solution and with the biologically active substance laminin, to form a water-based polymer dispersion incorporating biologically active molecules, using the following procedure.

An aqueous solution of laminin was prepared by dissolving laminin in water to reach a laminin concentration of about 100 μg/cm³. An aqueous solution of sodium chloride was then prepared by dissolving about 1.0 g sodium chloride in about 10 g of deionized water. About 1 g of the aqueous solution of laminin was mixed with about 3 g of the aqueous sodium chloride solution and the mixture was added in to a solution containing about 1.8 g of PLGA dissolved in about 12 g chloroform, to form the polymer dispersion.

Ultrasonication was used for preparing the dispersion. The duration of the process of ultrasonication (Sonic Dismembrator model 500, Fisher Scientific) was about 4 minutes, where about 2 second long pulses were alternated with about 2 seconds long stops, at amplitude of 30% and temperature of about 0° C. The resultant PGLA/water dispersion containing sodium chloride and laminin was then placed in the apparatus disclosed in WO 05/096744. The polymer dispersion was electropulled to form a resulting 3-dimensional oriented PLGA fiber. The length of the fibers was the same as the distance between the electrode and the collector , i.e., about 6 inches or 15 cm.

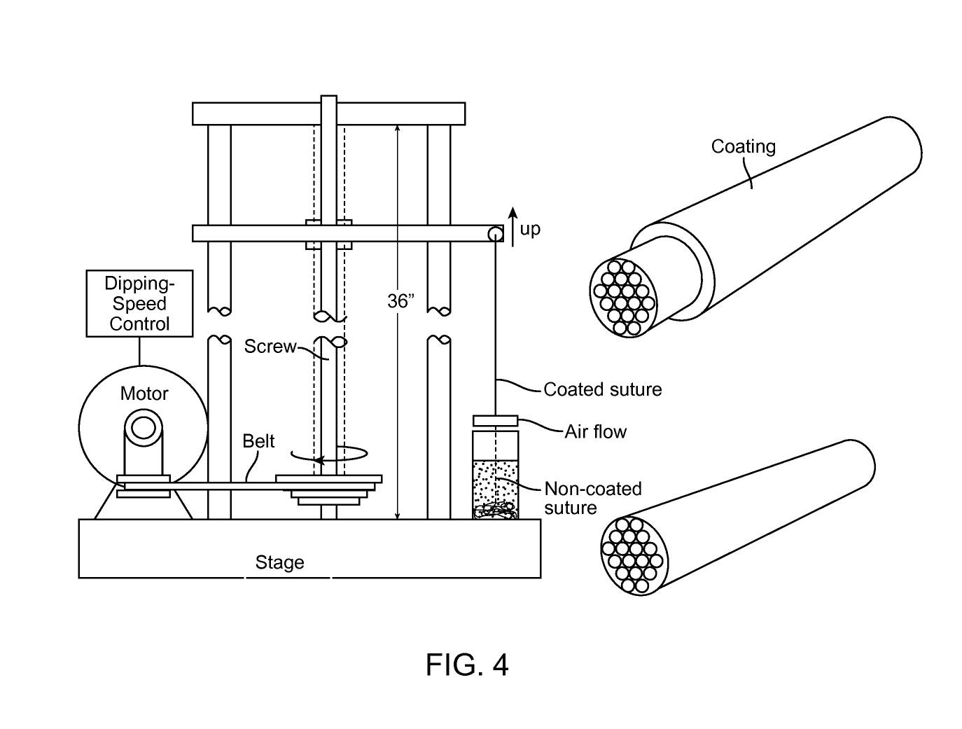

The non-coated fiber was placed in the apparatus shown in FIG. 4. The container containing the non-coated fiber was filled with a solution containing caprolactone/glycolide copolymer that forms the coat. The non-coated fiber was coated by pulling it through the coating solution and drying it using air flow.

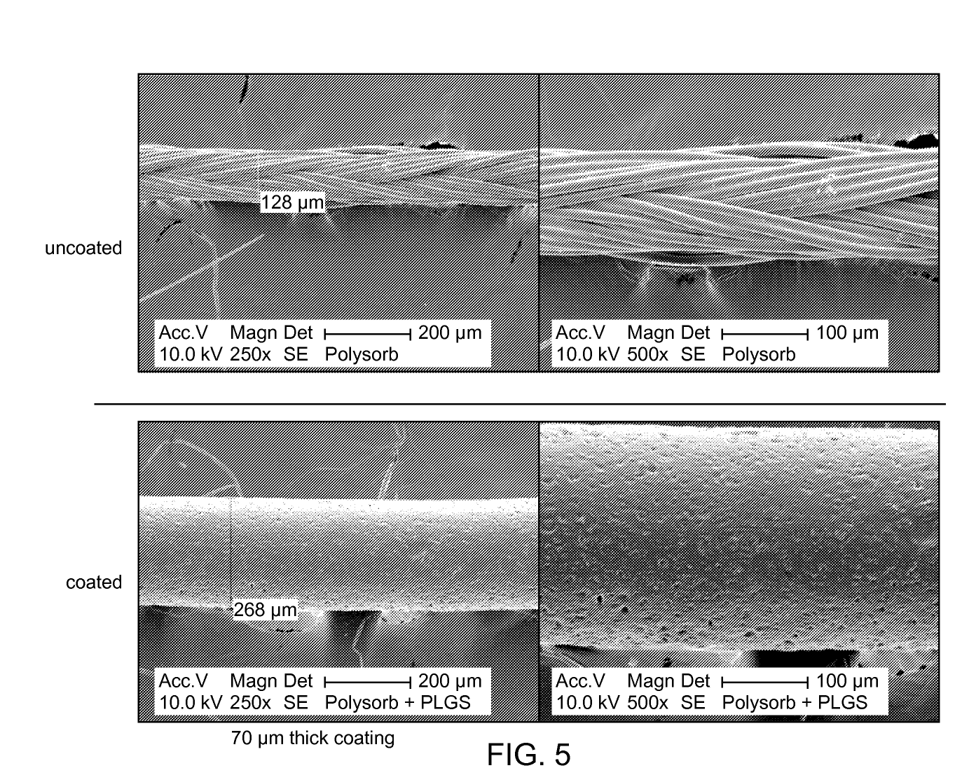

FIG. 5 shows the microphotographic images of the uncoated PLGA fiber and the PLGA fiber coated with PLGS formed as a result of the process described above. As can be seen, smooth, oriented, electropulled fibers have been produced that have a coating of about 70 μM.

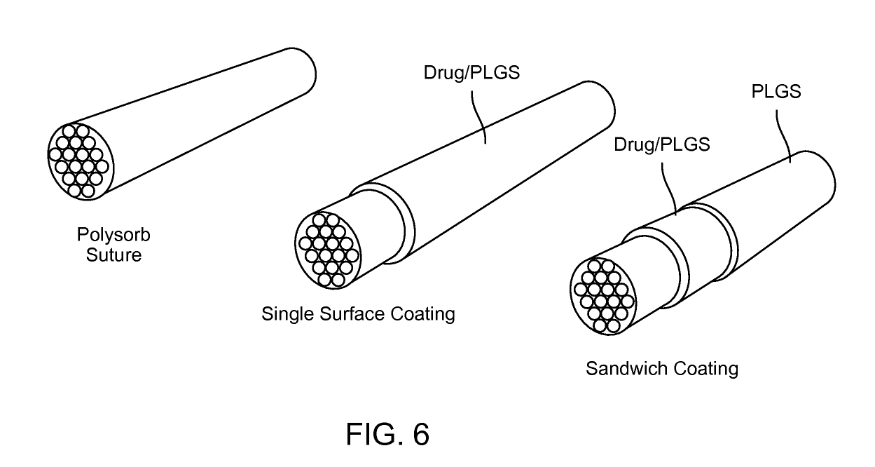

FIG. 6 illustrates a non-coated fiber, a coated fiber, and a sandwich coated fiber made using the methods described above.

While the invention has been particularly shown and described with reference to a preferred embodiment and various alternate embodiments, it will be understood by persons skilled in the relevant art that various changes in form and details can be made therein without departing from the spirit and scope of the invention. All printed patents and publications referred to in this application are hereby incorporated herein in their entirety by this reference.

We claim:

1. An endovascular device, comprising:
a polymer coil comprising a biocompatible and bioabsorbable polymer; and
a coating on the polymer coil wherein the coating reduces friction.

2. The apparatus of claim 1, wherein the biocompatible and bioabsorbable polymer is selected from the group consisting of polyglycolic acid (PGA), poly-L-lactic acid (PLLA), polycaprolactive, poly-L-lactide, polydioxanone, polycarbonates, polyanhydrides, polyglycolic acid/poly-L-lactic acid copolymers, and polyhydroxybutyrate/hydroxyvalerate copolymers, or combinations thereof.

3. The apparatus of claim 2, wherein the biocompatible and bioabsorbable polymer is a polyglycolic acid/poly-L-lactic acid copolymer.

4. The apparatus of claim 2, wherein the biocompatible and bioabsorbable polymer is PGA or PLLA.

5. The apparatus of claim 1, wherein the coating is selected from a group consisting of polylactide/polyglycolide copolymer (PLGs), caprolactone, calcium stearoyl lactylate, and caprolactone/glycolide copolymer, or combinations thereof.

6. The apparatus of claim 5, wherein the coating is PLGs.

7. The apparatus of claim 5, wherein the coating is calcium stearoyl lactylate.

8. The apparatus of claim 1, wherein the coating further comprises a drug.

9. The apparatus of claim 8, wherein the drug is a growth factor.

10. The apparatus of claim 9, wherein the growth factor is selected from the group consisting of vascular endothelial growth factor (VEGF), basic fibroblast growth factor (b-FGF), TGF, and PDGF, or mixtures thereof.

11. The apparatus of claim 10, wherein the growth factor is b-FGF.

12. The apparatus of claim 10, wherein the growth factor is VEGF and b-FGF.

13. The apparatus of claim 1, wherein the coating further comprises a radio-opaque material.

14. The apparatus of claim 1, wherein the coating further comprises a drug and a radio-opaque material.

15. The apparatus of claim 14, further comprising a second coating.

16. The apparatus of claim 15, wherein the second coating is PLGs.

17. An endovascular apparatus, the apparatus comprising:
a polymer coil comprising a biocompatible and bioabsorbable polymer; and
a sandwich coating on the polymer coil wherein the sandwich coating comprises at least a first coat and a second coat and wherein the sandwich coating reduces a friction coefficient of said apparatus.

18. The apparatus of claim 17, wherein the biocompatible and bioabsorbable polymer is selected from the group consisting of polyglycolic acid (PGA), poly-L-lactic acid (PLLA), polycaprolactive, poly-L-lactide, polydioxanone, polycarbonates, polyanhydrides, polyglycolic acid/poly-L-lactic acid copolymers, and polyhydroxybutyrate/hydroxyvalerate copolymers, or combinations thereof.

19. The apparatus of claim 18, wherein the biocompatible and bioabsorbable polymer is a polyglycolic acid/poly-L-lactic acid copolymer.

20. The apparatus of claim 18, wherein the biocompatible and bioabsorbable polymer is PGA or PLLA.
